(19) United States
(12) Patent Application Publication
(10) Pub. No.: US 2011/0117191 A1
(43) Pub. Date: May 19, 2011

(54) MIXTURE OF SUBSTANCES ON THE BASIS OF A MIXTURE OF ESSENTIAL OILS AND USE

(76) Inventor: Marianne Brøndlund, Harlev (DK)

(21) Appl. No.: 12/903,352

(22) Filed: Oct. 13, 2010

(30) Foreign Application Priority Data
Nov. 13, 2009 (DE) 10 2009 053 089.4

(51) Int. Cl. A61K 9/48; A61K 36/15; A61K 33/30; A61K 33/06; A61K 35/407; A61K 39/145; A61K 33/42; A61K 33/04; A61K 33/44; A61K 35/74; A61K 35/56; A61K 35/64; A61P 9/00; A61P 11/00; A61P 37/08; A61P 29/00; A61P 31/00

(52) U.S. Cl. 424/456; 424/770; 424/643; 424/682; 424/553; 424/451; 424/209.1; 424/601; 424/705; 424/125; 424/93.4; 424/520; 424/539

(57) ABSTRACT

The invention relates to a substance mixture on the basis of a mixture of essential oils, wherein the oil mixture contains juniper oil and turpentine oil, in particular turpentine oil of the duster pine type or substantially comprises them, and wherein the substance mixture preferably furthermore contains a liquid and/or dissolved active ingredient or a mixture of a plurality thereof and/or an active ingredient in powder form or a mixture of a plurality thereof.

The present invention relates to a mixture of substances on the basis of a mixture of essential oils and the use of a mixture of substances on the basic of a mixture of essential oils.

The use of essential oils in pharmaceuticals, in particular herbal pharmaceuticals, is known in the prior art. DE 39 02 981 A1, for instance, discloses the use of essential oils together with different vehicles on a patch. In this respects active ingredients should be produced for transdermal therapy. In DE 639 37 692 T2, the use of essential oils is proposed in combination with 1-menthol as an active dermal ingredient with a migraine-relieving effect. Salves and patches are named as preferred preparations. EP 0 800 827 B1 discloses the medical use of essential oils for the regulation of the blood platelet aggregation, either atone or in conjunction with a treatment with acetylsalicylic add. Blood platelet aggregation can be caused, for example, by the release of adrenalin or by smoking, with a treatment success being said to be achieved beyond the simple therapy with AAS with essential oils, in particular with blood platelet aggregation initiated by adrenalin.

Whereas, as just discussed, above all the dermal use of essential oils is emphasized in the prior art, DE 698 30 049 T2 discloses a medical or cosmetic preparation on the basis of essential oils, wherein such medical preparations are suitable for oral administration.

In the past few decades, an increased demand for herbal-based and natural medicines has been able to be generally recorded, frequently as a complement to conventional treatment. Such medicines are frequently low in side effects and are in accord with a lifestyle aware of nature, which is inter alia responsible for their increasing popularity among patients. Whereas the use of individual essential oils is known in herbal medicines, in particular in topically effective herbal medicines, it is now a goal of the present invention to combine various essential oils in remedies for oral administration and to provide a basis for new herbal remedies by this combination of the active herbal ingredients contained therein. Treatment successes should then be achieved, optionally in combination with further medicinal therapies, for infectious diseases, coughs, colds, bronchitis, sinusitis, allergic reactions, asthma, symptoms of the cardiovascular system, voiding disorders in prostate hyperplasia, wound healing problems, jaw problems, toothache, rheumatism and stomach ache.

Recovery should in this respect be assisted on a physical and mental level by the soothing odor, the warming properties and the generally sensory stimulating properties of the essential oils. This mixture of essential oils should in particular serve as a basis for the addition of a plurality of other active pharmaceutical ingredients and homeopathic substances.

This goal is achieved by a substance mixture in accordance with claim 1. Advantageous embodiments result from the dependent claims.

A substance mixture is accordingly provided in accordance with the invention on the basis of a mixture of essential oils, wherein the oil mixture contains juniper oil and turpentine oil, in particular turpentine oil of the duster pine type. In another embodiment, the oil mixtures essentially comprises juniper oil and turpentine oil, in particular turpentine oil of the duster pine type.

Juniper oil has the Latin name oleum junipers e baccarae; turpentine oil of the cluster-pine type oleum terebinthinae rectificat.

A non-synthetic remedy or a non-synthetic food supplement on the basis of the combination of these two essential oils can in particular be suitable for the alternative or complementary treatment of infections, symptoms such as asthma caused by allergic reaction or symptoms of the cardiovascular system, voiding disorders in prostate hyperplasia, wound healing problems, jaw problems, toothache, rheumatism and stomach ache. In this respect, an effect in cold complaints such as coughs, catarrh, sinusitis, headaches due to colds, bronchitis, pneumonia and similar is to be particularly emphasized. Optionally, a substance mixture on the basis of this mixture of juniper oil and turpentine oil can be used together with an influenza program from the prior ad.

The substance mixture is furthermore especially suitable as a medicine for use with influenza, optionally a disease in connection with an influenza epidemic, due to the speed and the strength of the onset of the effect. Inflammation effects are greatly minimized so that the unpleasant and potentially threatening strong symptoms. In acutely infected patients can be rapidly relieved.

The concentration of the ingredients of a substance mixture in accordance with the invention can be selected to be so strong that the length of the disease is considerably reduced. More detailed concentration information will be discussed in connection with the following embodiments. As soon as the body is complaint-free, the taking of the substance mixtures, optionally together with the influenza program from the prior art, should be stopped.

A substance mixture in accordance with the invention on the basis of a mixture of essential oils furthermore preferably includes a liquid or dissolved active ingredient or a mixture of a plurality thereof and/or an active ingredient in powder form or a mixture of a plurality thereof. Different active ingredients and excipients can thereby also be introduced into the substance mixture in accordance with the invention, whereby the effect of the oil mixture can be amplified or other different effects of substance mixtures in accordance with the invention can first be brought about.

In a preferred embodiment, a substance mixture in accordance with the invention is intended for oral administration, in particular as a food supplement. The effect of the substance mixture in accordance with the invention should develop on oral administration.

In a further preferred embodiment the substance mixture in accordance with the invention is present in apportioned form in the form of a capsule, preferably of a soft gelatin capsule, which can be administered orally. It can be ensured by the installation in a capsule that the desired dosage is exactly observed, that the substance mixture can be administered to patients with the desired strength of taste or odor, which is in particular desirable due to the intense organoleptic properties of juniper oil and turpentine oil, and/or that the uninhibited penetration of highly concentrated juniper extracts or pine extracts into the respiratory system is regulated, reduced or prevented. A soft gelatin capsule is therefore particularly advantageous since conventional plastic capsules can be dissolved by the inclusion of juniper oil and turpentine oil.

In a further preferred embodiment, the substance mixture in accordance with the invention is a food supplement or a medicine, in particular a natural herbal or homeopathic food supplement or medicine. Any additives are thereby also in accord with the non-synthetic character of the basis and together produce a purely natural substance mixture.

In a further embodiment, the basis, in a substance mixture in accordance with the invention, i.e. in the mixture of the essential oils, is juniper oil and turpentine oil: the mixture ratio of the oils is between approximately 7 to 3 and approximately 3 to 7, preferably between approximately 6 to 4 and approximately 4 to 6, and particularly preferably approximately 1 to 1, measured in parts by volume. Smaller deviations of the exact mixture ratio from 1:1 can be tolerated.

In a further embodiment, between approximately 10 mg and approximately 30 mg, preferably between approximately 15 mg and approximately 25 mg, and particularly preferably approximately 20 mg, juniper oil is contained in an orally administrable capsule or in another orally administrable unit of a substance mixture in accordance with the invention. The preferred content of turpentine oil results from the previously defined mixture ratio. These comparatively high dosages have proved to be particularly advantageous in connection with the occurrence of the above-described effects.

In a particularly preferred embodiment, the substance mixture in accordance with the invention on the basis of turpentine oil and juniper oil furthermore has a liquid or dissolved active ingredient or a mixture of a plurality thereof. These liquid or dissolved substances include individually non-synthetic remedies selected from the group of bacteria, nosodes, plant extracts and the body's own substances and active ingredients containing Zn²⁺ ions or Mg²⁺ ions.

Substances of natural origin, for example of plant origin, are to be understood as non-synthetic remedies, inter alia also homeopathic agents. In the doses used, they are preferably not subject to licensing, but are recognized in alternative medicine and are therefore used and tried and tested.

Suitable liquid or dissolved active ingredients or active ingredient mixtures individually include Hepar bovis, zinc, magnesium, crataegus, Spigella antheimia, Tonsillitis nosode, Myosotis avensis, Veronica officinalis, Teucrium scorodonia, Pinus sylvestris, Gentiana lutea, Equisetum hiemale (HAB 34), Smifax, Scrophularia nodosa, Calcium phosphoricum, Natrium sulfuricum, Fumaria officinalis, Levothyroxinum, Aranea diadema, Geranium robertanium, ethanol, Geranium robertainium, Nasturium officinale, Stapphylococcus-Nosode (Staphylococcus epidermitis, Staphylococcus haemolyticus, Stapphylococcus simulans), Streptococcus haemolyticus nosode, Otitis media nosode, tuberculosis nosode, diphtheria nosode, malaria nosode, Cimicifuga racemose, Strychnos nux vomica, serotonin, Ubichinon, Aconitum napellus, Lachesis mutus, Bryonia, Eupatorium perfoliatum, phosphorous, influenza nosode, Magnesium phosphoricum, Manganum phosphoricum, Natrium pyruvicum, Natrium oxalaceticum, Acidum citricum, Acidum cis-aconiticum, Barium oxalicum, Accidum succinicum, Acidum alpha-ketogiutaricum, Acidum succinicum, Acidum fumaricum, Acidum DL-maticum, Acium phosphoricum, Argentum nitricum, platinum, Sepia officinalis, Valeriana officinalis, Avena sativa, Ignatia injeel forte, Strychnos ignatil and Acidum arsenicosum. These active ingredients are combined in a further sequence as active ingredients of the group (I).

If these ingredients are present in a substance mixture in accordance with the invention, the arising product mixture can in particular be suitable for the treatment of patients with minor and serious infections of all types as well as of other cold diseases, in particular influenza, coughs, catarrh, headaches due to cold, sinusitis, bronchitis or pneumonia. This in particular applies in combination with individually selected substances from the groups (XI) or (XII) described in the following.

Further suitable liquid or dissolved active ingredients or active ingredient mixtures individually include Viscum album, Amica montana, Nicotiana tabacum, Hepar suls, Acidum arsenicosum, Ouabainum, Ranunculus bullbosus, Selenicereus grandiflorus, Nitroglycarinum, Kalium carbonicum, Kalmia latifolla, Spigelia anthelmia, Carbo vegetabilis, Acidum DL-malicum, Acidum sacrotacticum, Natrium oxalaceticum and Vena suis. These active ingredients are combined in a further sequence as active ingredients of the group (II).

If these components are present in a substance mixture in accordance with the invention the arising product mixture can in particular be suitable for treatment of patients with symptoms of the cardiovascular system. This in particular applies in combination with individually selected substances from the group (I) and/or with substances individually selected from the groups (XI) or (XII).

In a further embodiment, suitable liquid or dissolved active ingredients or active ingredient mixtures individually include Propolis, histamine, Mucosa nasalis suis, tetanus antitoxin, Ren suis, Hepar suls, zink valeriat, Apis mellifica, Apisinum, Urginea maritima, Kalum stibyltartaricum, Medulla spinails suis, Medulla oblongata suis. These active ingredients are combined in a further sequence as active ingredients of the group (III).

These active ingredients should above all be present in a substance mixture in accordance with the invention when the substance mixture should be used for the treatment of patients with symptoms caused by allergic reaction, in particular highly swollen insect bites, acute asthma attacks or spastic bronchitis with allergy. This in particular applies in combination with individually selected substances from the group (I) and/or with substances individually selected from the groups (XI) or (XIII).

In a preferred embodiment, in a substance mixture in accordance with the invention, the oil mixture used as a basis and the liquid or dissolved active ingredients or the corresponding active ingredient mixtures are present in a volume ratio of between approximately 2 to 8 and approximately 4 to 6, preferably from between approximately 25 to 75 and approximately 35 to 65, and particularly preferably from approximately 3 to 7.

If the substance mixture in accordance with the invention is provided in the form of a capsule, provision is made that a capsule in an embodiment contains between approximately 70 mg and approximately 200 mg, preferably between approximately 90 mg and approximately 180 mg, and particularly preferably between approximately 120 mg and approximately 150 mg of a liquid component. This liquid component essentially comprises the oil mixture used as the base and, optionally, the liquid or dissolved substances or substance mixtures.

Provision is made in a further preferred embodiment that a substance mixture in accordance with the invention furthermore has an active ingredient in powder form or a mixture of a plurality thereof in addition to the basis of juniper oil and turpentine oil and, optionally, the liquid or dissolved substances or substance mixtures. Active ingredients in powder form include non-synthetic ingredients, optionally herbal or homeopathic active ingredients and preferably individually inorganic ions, in particular calcium and magnesium, phosphorous and/or sulfur and their compounds, plant extracts, fats, nosodes, bacteria or carbon. The term non-synthetic remedy is defined as in connection with preceding embodiments.

In a preferred embodiment, active ingredients in powder form include individually one or more ingredients selected from the group Arnica montane, Calendula officinals, Hamamelis virginiana, Achillea millefollum, Atropa belladonna, Aconitum napellus, Mercurius solubills Hahnemanni, Hepar sutfuris, Chamomile recutita, Symphytum officinale, Beills parennis, Echinacea angustifolla, Echinaces purpurea, Hypericum perforatum, proteins, carbohydrates, calcium carbonate, magnesium carbonate, lactose, magnesium stearate, silica, microcrystalline cellulose, talc, polyvinyiptyrrolidone, Rhizoma Zingberi officinali Rose, Calcium, Anamirta cocculus, Conium maculatum, Ambra grisea and Petroleum rectificatum, Arnica, Gentians Lutea, Camomilla recucita, Juniperus cumunis and Atemisla Apsintium. These active ingredients are combined in a further sequence as active ingredients of the group (XI).

In a further embodiment, active ingredients in powder form individually include one or more active ingredients selected from the group Herba taraxati, Flos Sambucci, Herba Melissae, Herba Marubil albi, Herba Menthae crispae, Herba Gel, Radix inulae, Fructus Anisis, Herbs Agrimoniae, Rhizoma Valeriana, Semen Colae, Radix Gentianae, Cortex Grangulae, Fructus Juniperi, Flos Sambuci, Stigma Maidis, Herba Thymi, Herbs Salviae, Herbs Levistici, Rhizoma Velerianae, Herba Verbenae, Rhizoma Galarsgae, Folium Rosmarini, Rhizoma Rhei, Herbs Equiseti arvensis, Herbs Urticae, Herba Mentae Piperitae, Folium Menyanthis, Flos Chamomillae, Fructus Juniperi, Rhizoma Potentillae erectae, herba Centauril, Radix Sarsaparillae, Cortex Condurango, Cortex Quercus, Aim, Cartaegus oxycantha L., Samubucs nigra L., Viola tricolor L., Citrus aurantium L. ssp aurantium, Sophora japonica L., Herba Mellssae, Rhizoma Zingiberis, Herba Potentillae anserinae, Herba Marubii albi, Flos Crataegi oxycanthae, Canageen, Herba Menthae Peperitae, Herba Cardui benedicli, Rhizoma Valerianae, Herba Violas tricoloris, Folium Juglandis, Fructus Juniperis, Herba Thymi, Herba Urticae, Fructus juniperis, Herba Fumariae, Carrageen, Herba violae odoratae, Flos trifolli pratensis, Clematis recta, Herba Scolopendri, Rhizome valerianae, Cortex Chinchonae, Folium rosmarini, Cortex condurango, Fucus vesiculosus L. Sophora japonica L., Salvia officinalis L., Juniperus communis L., Trigonella foenum-graecum L. Linum usitaessimum L., Curcuma longa L., Chillea millefolium L., Viola tricolor L., Cola nitida (Vent) A, Chev, Argimonia eupatoral L. Cinchona succiruba Pavex Klotsch, Marsdenia cundurango Reichb. Fill., Quercus rubor L. Guajacum officinale L., Punica Granatum L., Rhizoma Gentiana Lutea L., Rhizoma Inula helenium L., Smilax aristolochiafolia Mlll., Angelica archangelica L., Rhadix Alpina officinarum Hance, Pimpinella saxifrage L., Rhizoma Rheum palmatum L., Potentilla erecta L., Viola odorata L., Urtica diolca L., Viola tricolor L., Euphrasia officinalis L., Valeriana officinalis L., Flos Juglans regia L., Malva syivestris L., Althaea officinalis L. Plantago lanceolata L., Foeniculum vulgara Mill., Matricaria chamomilla L., Phaseolus vulgatis L., Salvia officinalis L., Berberis vulgaris L., Menthax piperita L., Taraxacum officinalis Web., Valeriana officinalis L., Trigonella foenum-graecum L., Lavandula angustifolia Mill., Melissa officinalis L., Centaurium erythaea Rafin., Cnicus benedictus L., Humulus lupus L., Chondurs crispus Stackh. (Irish moss), Citrus aurantium L. ssp aurantium, Quercus petrae Liebl., Nasturtium officinale R.Br, Taraxacum officinale Web, Primula veris L., Levisticum officinaie Koch, Smiley aristolochiafolia Mill., Rhadix lmperatoria asthruthium L., Euphorbia pilulifera, Jaquin, Gallium odaratum L. Scop, Verbena officinalis L., Hamamelis virginiana L., Viola odoratum L., Teucrium scordium L., Origanum vulgare L., Euphrasia officinalis L., Crataegus oxycantha L., Hederahelix L., Equiselum arvense L., Juniperus communis L., Valeriana officinalis L., Viola tricolor L., Crataegus oxycantha L., Menthax piperita L., Matricaria chamollia L., Sophora japonica L., Potentilla erecta Raeusch, Melissa officinalis L., Verba scumphiomoides L., Artemisia absinthum L., Menyanthis trifoliata L., Fumaria officinalis L., Berberis vulgaris L., Salvia officinalis L., Rhamnus franguia L., Jugian regia L. and Cnicus benedictus L. These active ingredients are combined in a further sequence as active ingredients of the group (XII).

If these components are present in a substance mixture in accordance with the invention, the arising product mixture can in particular be suitable for treatment of patients with minor and serious infections of any kind as well as other cold diseases, in particular influenza, cough, catarrh, headaches due to cold, sinusitis, bronchitis or pneumonia or with symptoms of the cardiovascular system. This in particular applies in combination with individually selected substances from the groups (I), (II) or (XI).

In a further embodiment, active ingredients in powder form individually include one or more active ingredients selected from the groups Acidum sillcicum, Histaminum muriaticum, Apis mellifica, Apisinum, Urginla, Maritima and Callum Stibyltartaricum. These active ingredients are combined in a further sequence as active ingredients of the group (XIII).

These active ingredients should above all be present in a substance mixture in accordance with the invention when the substance mixture should be used for the treatment of patients with symptoms caused by allergic reaction, in particular highly swollen insect bites, acute asthma attacks or spastic bronchitis with allergy. This in particular applies in combination with individually selected substances from the groups (I), (III) or (XI).

All those of the substances discussed in connection with the preceding embodiments can be used, provided they are homeopathic substances, in any desired homeopathic potencies, preferably, however, the potencies D1, D2, D3, D4, D6, D8, D10 and D30 are used.

Provision is made in an embodiment for a dosage, optionally a capsule, of a substance mixture in accordance with the invention, that it contains between approximately 200 mg and approximately 400 mg, preferably between approximately 250 mg and approximately 360 mg, and particularly preferably approximately 300 mg of the active ingredient or ingredients in powder form.

The invention further relates to the use of a substance mixture in accordance with the invention for the manufacture of a medicine or of a food supplement. In particular of a non-synthetic, herbal or homeopathic medicine. The therapy can in this respect be used as a sole therapy or as an assisting therapy for conventional orthodox medicine or other alternative medicine therapies. It should preferably only be carried out during the disease. The dosage and the duration of the therapy should generally be adapted to the gravity of the disease.

Provision is made in a preferred embodiment that in the acute stage of the complaints, 4×1 capsules should be taken for 4 days (in the morning, mid-day, in the evening, at night); then 3×1 capsules for 3 days (in the morning, mid-day, in the evening) and then 2×1 capsules for 2 days (in the morning and in the evening). Provision is, however, made in another embodiment that the dosage can be reduced with respect to the preceding embodiment if the symptoms are not all that severe or if an improvement already occurs at an early time. The administration of 3×1 capsules for 3 days (in the morning, mid-day and in the evening) and then 2×1 capsules for 2 days (in the morning and in the evening) can accordingly be sufficient. The administration of 1 to 2 capsules at night at the start of a cold or of a sore throat can also be effective. The properties of the capsules and the quantities and the ratios of the active ingredients contained were discussed in connection with the preceding embodiments. The resulting dosages serve as a reference for promising therapies with the aid of a substance mixture in accordance with the invention.

In a preferred embodiment, the substance mixture in accordance with the invention is used for the manufacture of an orally administrable medicine. The medicine should be suitable in a further embodiment for the therapy of minor and serious infections of any kind as well as of other cold diseases. in particular be intended for influenza, cough, catarrh, headaches due to colds, sinusitis, bronchitis or pneumonia, and in other embodiments for the therapy of symptoms caused by allergic reaction such as asthma and/or symptoms of the cardiovascular system.

In another embodiment, a medicine produced in accordance with the invention on the basis of a substance mixture in accordance with the invention should be suitable for use in flu-like infections, optionally in such a disease in combination with an influenza epidemic. The advantageous use is here particularly due to the speed and the strength of the effect onset, wherein inflammation effects are highly minimized and the unpleasant and potentially threatening, serious symptoms can be changed rapidly in acutely infected patients.

In a further embodiment, a substance mixture in accordance with the invention can be used as a medicine or as a food supplement in the treatment of voiding disorders in prostate hyperplasia, wound heating problems, jaw problems, toothache, rheumatism and/or stomach aches.

The invention further relates to the use of a substance mixture in accordance with the invention containing one or more ingredients, preferably the totality of the ingredients, from the active ingredient groups I and II and XI and XIII for the treatment of patients with symptoms caused by allergic reaction, in particular highly swollen insect bites, acute asthma attacks or spastic bronchitis in allergies and other allergically caused reactions or symptoms. In this respect, the substance mixture in accordance with the invention should be used alone or as an assisting therapy.

The present invention further relates to the use of a substance mixture in accordance with the invention containing one or more ingredients, preferably the totality of the ingredients, from the active ingredient groups I and III and XI and XII for the treatment of patients with symptoms of the cardiovascular system. In this respect, the substance mixture in accordance with the invention should be used as the sole or as an assisting therapy.

Further details and advantages of the invention result from the following embodiments.

### EXAMPLE 1

### Oil Mixture as the Basis

100 ml Oleum Juniperi e Baccarae, PH.EUR.6.0, juniper oil, Article #PZW 2282095 from Caesar & Lorenz GmbH, D-40721 Mien are mixed with 100 ml Oleum Terebinthinae rectificat., PH.EUR.6.0, turpentine oil of the duster pine type, Article #PZW 1786831 from Caesar & Lorenz GmbH, D-40721 Hilden, and are stirred well.

The oil mixture results from this which forms the basis for a substance mixture in accordance with the invention.

### EXAMPLE 2

### Manufacture of a Mixture of a Plurality of Liquid or Dissolved Active Ingredients

The quantities of the following ingredients set forth are mixed well and represent the basis for an ampoule mixture.

| Dosage | Product | Manufacturer |
|---|---|---|
| 25 ampoules @1.1 ml | Gripp-Heel | Biologische Heilmittel |
| | | Heel GmbH |
| 10 ampoules @ 1 ml | Hepar/Stannum II | WALA Heilmittel GmbH |
| 10 ampoules @ 1 ml | Hepar-Magnesium | Weleda AG |
| | Potency D4 | |
| 10 ampoules @ 1 ml | Malaria Nos. D6-D30 | Meripharm GmbH |
| 10 ampoules @ 1 ml | Tuberculocidinum | Meripharm GmbH |
| | Klebs Nos. D6 | |
| 10 ampoules @ 1 ml | Diphterium Nos. D30 | Meripharm GmbH |
| 10 ampoules @ 1.1 ml | Cranolinum | Biologische Heilmittel |
| | | Heel GmbH |
| 20 ampoules @ 1.1 ml | Tonsillitis-Nosode- | Biologische Heilmittel |
| | Injeel | Heel GmbH |
| 20 ampoules @ 1.1 ml | Lymphomyosot | Biologische Heilmittel |
| | | Heel GmbH |
| 20 ampoules @ 1.1 ml | Staphylococus-Injeel | Biologische Heilmittel |
| | | Heel GmbH |
| 20 ampoules @ 1.1 ml | Streptococus | Biologische Heilmittel |
| | haemolyticus-Injeel | Heel GmbH |
| 20 ampoules @ 1.1 ml | Otitis media-Nosode- | Biologische Heilmittel |
| | Injeel | Heel GmbH |
| 20 ampoules @ 1.1 ml | Cimicifuga-Injeel | Biologische Heilmittel |
| | forte S | Heel GmbH |
| 20 ampoules @ 1.1 ml | Zitronensäurezyklus- | Biologische Heilmittel |
| | Heel [Citric Acid | Heel GmbH |
| | Cycle Heel] | |
| 20 ampoules @ 1.1 ml | Para-Benzochinon- | Biologische Heilmittel |
| | Injeel forte | Heel GmbH |
| 20 ampoules @ 1.1 ml | Acidum fumaricum- | Biologische Heilmittel |
| | Injeel forte | Heel GmbH |
| 20 ampoules @ 1.1 ml | Ubichinon-Injeel | Biologische Heilmittel |
| | forte | Heel GmbH |
| 20 ampoules @ 1.1 ml | Nux vomica-Injeel | Biologische Heilmittel |
| | S | Heel GmbH |
| 20 ampoules @ 1.1 ml | Serotonin-injeel | Biologische Heilmittel |
| | | Heel GmbH |
| 20 ampoules @ 1.1 ml | Neuro-Injeel | Biologische Heilmittel |
| | | Heel GmbH |
| 10 ampoules @ 1.1 ml | Arsenicum Album- | Biologische Heilmittel |
| | Injeel S | Heel GmbH |
| 7 drops | Bach flower rescue | Nelsons Pharmacy |
| | drops | London |

The mixture created represents an embodiment for the active ingredient mixture (I) as defined in the general part of the patent description.

### EXAMPLE 3

### Manufacture of a Mixture of a Plurality of Liquid or Dissolved Active Ingredients

The following ingredients are mixed to an ampoule mixture from example 2 and the mixture created is mixed well.

| Dosage | Product | Manufacturer |
|---|---|---|
| 10 ampoules @ 1.1 ml | Cor suis compositum N | Biologische Heilmittel |
| | | Heel GmbH |
| 10 ampoules @ 1.1 ml | Arteria suis-injeel forte | Biologische Heilmittel |
| | | Heel GmbH |
| 10 ampoules @ 1.1 ml | Vena suis-injeel | Biologische Heilmittel |
| | | Heel GmbH |
| 10 ampoules @ 1.1 ml | Aorta suis-injeel forte | Biologische Heilmittel |
| | | Heel GmbH |

This mixture represents an embodiment for the active ingredient mixture (II) as defined in the general part of the patent description.

### EXAMPLE 4

### Manufacture of a Mixture of a Plurality of Liquid or Dissolved Active Ingredients

The following ingredients are furthermore mixed to the ampoule mixture from example 2 and the mixture created is mixed well.

| Dosage | Product | Manufacturer |
|---|---|---|
| 4 g to 100 g EtOH 96% | Propolis | |
| 20 ampoules @ 1.1 ml | Apis-injeel S | Biologische Heilmittel |
| | | Heel GmbH |
| 20 ampoules @ 1.1 ml | Histamin-injeel | Biologische Heilmittel |
| | | Heel GmbH |
| 20 ampoules @ 1.1 ml | Pollens-injeel forte | Biologische Heilmittel |
| | | Heel GmbH |
| 20 ampoules @ 1.1 ml | Mucosa nasalis suis- | Biologische Heilmittel |
| | Injeel | Heel GmbH |
| 20 ampoules @ 1.1 ml | Medulla oblongata | Biologische Heilmittel |
| | suis-injeel | Heel GmbH |
| 20 ampoules @ 1.1 ml | Medulla spinalis suis- | Biologische Heilmittel |
| | Injeel | Heel GmbH |
| 20 ampoules @ 1.1 ml | Tetanus antitoxin | Biologische Heilmittel |
| | | Heel GmbH |
| 20 ampoules @ 1.1 ml | Ren suis-injeel | Biologische Heilmittel |
| | | Heel GmbH |
| 20 ampoules @ 1.1 ml | Hepar suis-injeel | Biologische Heilmittel |
| | | Heel GmbH |
| 20 ampoules @ 1.1 ml | Zincum valerianicum- | Biologische Heilmittel |
| | injeel | Heel GmbH |

This mixture represents an embodiment for the active ingredient mixture (III) as defined in the general part of the patent description.

### EXAMPLE 5

### Manufacture of a Mixture of Active Ingredients in Powder Form

The following ingredients are finely pounded and ground in the dosages set forth and are subsequently mixed wall.

| Dosage | Product | Manufacturer |
|---|---|---|
| 23 doses @ 50 tablets | Traumeel S | Biologische Heilmittel |
| | | Heel GmbH |
| 3 doses @ 120 tablets | VermiCAlite plus | Staufen Göppingen |
| 1 dose @ 200 tablets | Carbo animalis | DHU |
| (100 mg) | | |
| 1 dose @ 200 tablets | Carbo vegetabilis | DHU |
| (100 mg) | | |
| 1.5 sachets @ 96 mg | Wolle's No. 370 | Wolle Nature System |
| 2 doses @ 100 tablets | Vertigoheel | Biologische Heilmittel |
| | | Heel GmbH |
| 1 dose | Knattwigast | Pharmazeutische Fabrik |
| | | Kattwiga |
| 1 dose @ 10 g | Arnica D12 Globuli | DHU |

This mixture represents an embodiment for the active ingredient mixture (XI) as defined in the general part of the patent description.

### EXAMPLE 6

### Manufacture of a Further Mixture of Active Ingredients in Powder Form

The following further ingredients are ground with the ingredients from Example 5 in addition to the mixture from Example 5:

| Dosage | Product | Manufacturer |
|---|---|---|
| 4 doses | Wolle's No. 1781 | Wolle Nature System |
| 4 doses | Wolfe's No. 1778 | Wolle Nature System |
| 4 doses | Wolle's No. 1748 | Wolle Nature System |
| 4 doses | Wolle's No. 1752 | Wolle Nature System |
| 4 doses | Wolle's No. 1706 | Wolle Nature System |
| 4 doses | Wolle's No. 1722 | Wolle Nature System |
| 4 doses | Wolle's No. 1731 | Wolle Nature System |
| 4 doses | Wolle's No. 1703 | Wolle Nature System |
| 4 doses | Wolle's No. 1784 | Wolle Nature System |
| 4 doses | Wolle's No. 1724 | Wolle Nature System |
| 4 doses | Wolle's No. 1702 | Wolle Nature System |
| 4 doses | Wolle's No. 1741 | Wolle Nature System |

This mixture represents an embodiment for the active ingredient mixture (XII) as defined in the general part of the patent description.

### EXAMPLE 7

### Manufacture of a Further Mixture of Active Ingredients in Powder Form

The following further ingredients are ground with the ingredients from Example 5 added to the powder mixture from Example 5:

| Dosage | Product | Manufacturer |
|---|---|---|
| 1 dose @ 100 tablets | Silicic acid tablets PZN | Cosmochema |
| (100 mg) | 4400170 | |
| 1 dose @ 100 tablets | Histaminum Muriaticum | DHU |
| (100 mg) | | |
| 1 dose @ 100 tablets | Pollens | DHU |
| (100 mg) | | |
| 1 dose @ 100 tablets | Apis mellifica | DHU |
| (100 mg) | | |

This mixture represents an embodiment for the active ingredient mixture (XIII) as defined in the general part of the patent description.

### EXAMPLE 8

### Substance Mixture for the Treatment of Infections

6 ml of the oil mixture from Example 1 are mixed with 14 ml of the ampoule mixture from Example 2 and 300 mg of the powder mixture from Example 6 and are mixed well. The created substance rnbcture is welded in a soft gelatin capsule and serves the administration by the patient. Such a capsule consequently contains roughly 4 drops of juniper oil corresponding to approximately 20 mg.

### EXAMPLE 9

### Substance Mixture for the Treatment of Symptoms Caused by Allergic Reactions

6 ml of the all mixture from Example 1 are mixed with 14 ml of the ampoule mixture from Example 3 and 300 mg of the powder mixture from Example 7 and are mixed well. The created substance mixture is welded in a soft gelatin capsule and serves the administration by the patient. Such a capsule consequently contains roughly 4 drops of juniper oil corresponding to approximately 20 mg.

### EXAMPLE 10

### Recipe for the Treatment of Symptoms of the Cardiovascular System

6 ml of the oil mixture from Example 1 are mixed with 14 ml of the ampoule mixture from Example 4 and 300 mg of the powder mixture from Example 8 and are mixed well. The created substance mixture is welded in a soft gelatin capsule and serves the administration by the patient. Such a capsule consequently contains roughly 4 drops of juniper oil corresponding to approximately 20 mg.

1. A substance mixture on the basis of a mixture of essential oils, wherein the oil mixture contains juniper oil and turpentine oil.

2. A substance mixture in accordance with claim 1, wherein the turpentine oil is turpentine oil of the cluster pine type.

3. A substance mixture in accordance with claim 1, wherein the oil mixture essentially comprises juniper oil and turpentine oil.

4. A substance mixture in accordance with claim 1, wherein the substance mixture is intended for oral administration.

5. A substance mixture in accordance with claim 4, wherein the substance mixture is present in the form of an orally administrable capsule.

6. A substance mixture in accordance with claim 5, wherein the capsule is a soft gelatin capsule.

7. A substance mixture in accordance with claim 5, wherein a capsule contains between approximately 10 and approximately 30 mg juniper oil.

8. A substance mixture in accordance with claim 7, wherein a capsule contains between approximately 16 and approximately 25 mg juniper oil.

9. A substance mixture in accordance with claim 8, wherein a capsule contains approximately 20 mg juniper oil.

10. A substance mixture in accordance with claim 1, wherein the substance mixture is a food supplement.

11. A substance mixture in accordance with claim 10, wherein the food supplement is a natural, herbal or homeopathic food supplement.

12. A substance mixture in accordance with claim 1, wherein the substance mixture is a medicine.

13. A substance mixture in accordance with claim 12, wherein the medicine is a natural, herbal or homeopathic medicine.

14. A substance mixture in accordance with claim 1, wherein the oil mixture contains juniper oil and turpentine oil in a volume ratio of between approximately 7:3 and approximately 3:7.

15. A substance mixture in accordance with claim 14, wherein the oil mixture contains juniper oil and turpentine oil in a volume ratio of between approximately 8:4 and approximately 4:8.

16. A substance mixture in accordance with claim 16, wherein the oil mixture contains juniper oil and turpentine oil in a volume ratio of appoximately 1:1.

17. A substance mixture in accordance with claim 1, further containing a liquid or dissolved active ingredient or a mixture of a plurality thereof.

18. A substance mixture in accordance with claim 17, wherein the liquid or dissolved active ingredients include non-synthetic medicines selected from the group of bacteria, nosodes, plant extracts and the body's own substances, and active ingredients containing Zn²⁺ ions or Mg²⁺ ions.

19. A substance mixture in accordance with claim 18, wherein the liquid or dissolved active ingredients or active ingredient mixtures include Hepar bovis, zinc, magnesium, Crataegus, Spigelia antheimia, Tonsillitis-Nosode, Myosolis avensis, Veronica officinalis, Teucrium scorodonia, Pinus sylvestris, Gentiana lutea, Equisetum hiemale (HAB 34), Smifax, Scrophularia nodosa, Calcium phosphoricum, Natrium sulfuricum, Fumaria officinalis, Levothyroxinum, Aranea diadema, Geranium robertanium, ethanol, Geranium robertanium, Nasturlum officinale, Ferrum jodatum, Stapphylococcus nosode (Staphylococcus epidermitis, Staphylococcus haemolyticus, Stapphylococcus simulans), Streptococcus haemolyticus nosode, Otitis media nosode, tuberculosis nosode, dipththeria nosode, malaria nosode, Cimicifuga racemosa, Strychnos nux vomica, serotonin, Ubichinon, Aconitum napellus, Lachesis mutus, Bryonia, Eupatorium perfoliatum, phosphorous, Influenza vaccine nosode, Magnesium phosphoricum, Manganum phosphoricum, Natrium pyruvicum, Natrium oxalaceticum, Acidum citricum, Acidum cis-aconiticum, Barium oxalicurn succinicum, Acidum alpha-ketoglutaricum, Acidum succinicum, Acidum fumaricum, Acidum DL-mallcum, Acidum phosphoricum, Argentum nitricum, platinum, Sepia officinalis, Valeriana officinalis, Avena sativa, Strychnos ignatii or Acidum arsenicosum.

20. A substance mixture in accordance with claim 18, wherein the liquid or dissolved active ingredients or active ingredient mixtures include Cor suis, Arteria suis, Vena suis and/or Aorta suis.

21. A substance mixture in accordance with claim 18, wherein the liquid or dissolved active ingredients or active ingredient mixtures include Propolis, Apis Forte, Histaminum Muriaticum, Pollens Forte, Muscosa Nasalis Forte, Medulla Oblongata, Medulla Spinalis, Tetanus Antitoxin, Ren Suis, Hepar Suis and/or Zinkum Valerianum

22. A substance mixture in accordance with claim 18, wherein the oil mixture and the liquid or dissolved active ingredients or active ingredient mixtures are present in a ratio of between approximately 2:6 and approximately 4:6.

23. A substance mixture in accordance with claim 22, wherein the oil mixture and the liquid or dissolved active ingredients or active ingredient mixtures are present in a ratio of between approximately 22:75 and approximately 35:65.

24. A substance mixture in accordance with claim 22 wherein the ratio of the oil mixture and the liquid or dissolved active ingredient or active ingredient mixture is at approximately 3:7.

25. A substance mixture in accordance with claim 18, wherein the substance mixture is present in the form of an orally administrable capsule and wherein a capsule contains between approximately 70 and approximately 200 mg of a liquid component substantially comprising the off mixture and the liquid or dissolved active ingredients.

26. A substance mixture in accordance with claim 25, wherein a capsule contains between approximately 90 and approximately 180 mg of a liquid component substantially comprising the oil mixture and the liquid or dissolved active ingredients.

27. A substance mixture in accordance with claim 26, wherein a capsule contains between approximately 120 and approximately 150 mg of a liquid component substantially comprising the oil mixture and the liquid or dissolved active ingredients.

28. A substance mixture in accordance with claim 1, further containing an active ingredient in powder form or a mixture of a plurality thereof.

29. A substance mixture in accordance with claim 28, wherein the active ingredients in powder form include inorganic ions, in particular calcium and magnesium, phosphorous and/or sulfur and their compounds, plant extracts, fats, nosodes, bacteria or carbon.

30. A substance mixture in accordance with claim 29, wherein the active ingredients or active ingredient mixtures in powder form include Arnica montane, Calendula officinalis, Hamamells virginiana, Achillea millefollum, Atropa belladonna, Aconitum napellus, Mercurius solubllis Hahnemannl, Hepar sulfuris, Chamomilla recutita, Symphytum officinale, Bellis parennis, Echinacea angustifolia, Echinacea purpurea, Hypericum perforatum, proteins, carbohydrates, calcium carbonate, magnesium carbonate, lactose, magnesium stearate, silica, microcrystalline cellulose, talc, polyvinylprynoildone, Rhizoma Zinglberi officinali Rose, calcium, Anamirta cocculus, Conium maculatum, Ambra grisea and Petroleum rectificatum, Arnica, Gentiana Lutea, Camomlila recucita, Juniperus cumunis and/or Atemisia Apsintlum, and optionally additionally include Herba taraxati, Flos Sambucci, Herba Melissae, Herba Marubil albi, Herba Menthae crispae, Herba Gei, Radix lnulae, Fructus Anisis, Herba Agrimoniae, Rhizoma Valeriana, Semen Colae, Radix Gentianae, Cortex Grangulae, Fructus Juniperi, Flos Sambuci, Stigma Maidis, Herba Thymi, Herba Selviae, Herba Levistici, Rhizoma Velerianae, Herba Verbenae, Rhizoma Galangae, Folium Rosmarini, Rhizoma Rhei, Herba Equiseti arvensis, Herba Urticae, Herba Mantae Piperitae, Folium Menyanthis, Flos Chamomillae, Fructus Juniperi, Rhizoma Potentillae erectae, herba Centauril, Radix Sarsaparillae, Cortex Condurango, Cortex Quercus, Alm, Cartaegus oxycantha L., Samubucs nigra L., Viola tricolor L., Citrus aurantium L. sap aurantium, Sophora Japonica L., Herba Melissae, Rhizoma Zinglberis, Herba Potentillae anserinee, Herba Marubli albi, Flos Crataegi oxycanthae, Carrageen, Herba Menthae Peperitae, Herba Cardui benedicti, Rhizoma Valerlanae, Herbs Violae tricoloris, Folium Juglandis, Fructus Juniperis, Herba Thymi, Herba Urticas, Fructus juniperis, Herba Fumariae, Carrageen, Herba violae odoratae, Flos trifolli pratensis, Clematis recta, Herba Scolopendri, Rhizoma valerlanae, Cortex Chinchonae, Folium rosmarini, Cortex condurango, Fucus vesiculosus L. Sophora japonica L, Salvia officinalis L., Juniperus communis L., Trigonella foenum-graecum L. Linum usitatlesimum L., Curcuma longa L., Chilea millefolium L., Viola tricolor L., Cola nitida (Vent) A, Chev, Argimonia eupatorai L. Cinchona succiruba Pavex Klotsch, Marsdenia cundurango Reichb. Fill. Quercus rubor L., Guajacum officinale L., Punica Granatum L., Rhizoma Gentiana Lutea L., Rhizoma lnuia helenium L., Smilax aristoiochiafolia Mill., Angelica archangelica L., Rhadix Alpina officinarum Hance, Pimpinella saxifrage L., Rhizoma Rheum palmatum L., Potentilla erecta L., Viola odorata L., Urtica diolca L., Viola tricolor L., Euphrasia officinalis L., Valerlana officinalis L., Flos Juglans regia L., Malva sylvestris L., Althaea officinalis L., Plantago lanceolata L., Foeniculum vulgara Mill., Matricaria chamomilla L., Phaseolus vulgatis L., Salvia officinalis L., Berberis vulgaris L., Menthax piperita L., Taraxacum officinalis Web., Valeriana officinalis L., Trigoneila foenum-graecum L., Lavandula angustifolia Mill., Melissa officinalis L., Centaurium erythraea Rafin., Onicus benedictus L., Humulus lupus L., Chondurs crispus Stackh. (Irish moss), Citrus aurantium L. asp aurantium, Quercus petrae Liebl., Nasturcium officinale R.Br, Taraxacum officinale Web, Primula veris L., Levisticum officinale Koch, Smilay aristolochiafolia Mill., Rhadix lmperatoria asthruthium L., Euphorbia piiulifera, Jaquin, Gallium odaratum L. Scop, Verbena officinalis L., Hamamelis virginiana L., Viola odoratum L., Teucrium scordium L., Origanum vulgare L., Euphrasia officinalis L., Crataegus oxycantha L., Hederahelix L., Equisetum arvense L., Juniperus communis L., Vaieriana officinalis L., Viola tricolor L., Crataegus oxycantha L., Menthax piperita L., Matricaria chamoilla L., Sophora japonica L., Potentilla arecta Raeusch, Melissa officinalis L., Verbascumphlomoides L., Artemisia absinthum L., Menyanthis trifoliata L., Fumaria officinalis L., Berberis vulgaris L., Salvia officinalis L., Rhamnus frangula L., Juglan regia L. and Cnicus benedictus L.

31. A substance mixture in accordance with claim 30, wherein the active ingredients or active ingredient mixtures in powder form include Silicea, Histaminum Muriaticum, Pollens and Apis mellificia

32. A substance mixture in accordance with claim 28, wherein the substance mixture is present in apportioned form in the form of an orally administrable capsule and wherein a capsule contains between approximately 200 mg and approximately 400 mg active ingredients in powder form.

32. A substance mixture in accordance with claim 32, wherein a capsule contains between approximately 250 mg and approximately 350 mg active ingredients in powder form.

33. A substance mixture in accordance with claim 33, wherein a capsule contains approximately 300 mg active ingredients in powder form.

35. Use of a substance mixture in accordance with claim 1 for oral administration to patients.

36. Use in accordance with claim 35, wherein the oral administration serves the treatment of infections.

37. Use in accordance with claim 38, wherein infections include cold diseases selected from the group of influenza infections, cough, catarrh, headaches due to colds, sinusitis, bronchitis or pneumonia.

38. Use in accordance with claim 35, wherein the oral administration serves the treatment of symptoms due to allergic reaction.

39. Use in accordance with claim 35, wherein the oral administration serves the treatment of diseases of the cardiovascular system.

40. Use in accordance with claim 35, wherein the oral administration serves the treatment of diseases selected from the group of voiding disorders in prostate hyperplasia, wound healing problems, jaw problems, toothache, rheumatism and stomach ache.

41. Use in accordance with claim 35, wherein the oral administration serves the sole treatment of diseases.

42. Use in accordance with claim 35, wherein the oral administration serves the complementary treatment of diseases.

43. Use of a substance mixture in accordance with claim 21 for the oral administration to patients for the treatment of symptoms caused by allergic reaction.

44. Use in accordance with claim 43 for the treatment of symptoms caused by allergic reactions selected from the group of highly swollen insect bites, acute asthma attacks and spastic bronchitis in allergy.

45. Use in accordance with claim 43, wherein the oral administration serves the sole treatment of diseases.

46. Use in accordance with claim 43, wherein the oral administration serves the complementary treatment of diseases.

47. Use of a substance mbdure in accordance with claim 31 for the oral administration to patients for the treatment of symptoms caused by allergic reaction.

48. Use in accordance with claim 47 for the treatment of symptoms caused by allergic reactions selected from the group of highly swollen insect bites, acute asthma attacks and spastic bronchitis in allergy.

49. Use in accordance with claim 47, wherein the oral administration serves the sole treatment of diseases.

50. Use in accordance with claim 47, wherein the oral administration serves the complementary treatment of diseases.

51. Use of a substance mixture in accordance with claim 20 for the oral administration to patients for the treatment of diseases of the cardiovascular system.

52. Use in accordance with claim 51, wherein the oral administration serves the sole treatment of diseases.

53. Use in accordance with claim 51, wherein the oral administration serves the complementary treatment of diseases.

54. Use of a substance mixture in accordance with claim 30 for the oral administration to patients for the treatment of diseases of the cardiovascular system.

55. Use in accordance with claim 64, wherein the oral administration serves the sole treatment of diseases.

56. Use in accordance with claim 54, wherein the oral administration serves the complementary treatment of diseases.
